(19) United States
(12) Patent Application Publication
(10) Pub. No.: US 2025/0296006 A1
(43) Pub. Date: Sep. 25, 2025

(54) VIBROTACTILE CUDDLE DEVICE

(71) Applicant: Gregory Scott Bishop, Irvine, CA (US)

(72) Inventor: Gregory Scott Bishop, Irvine, CA (US)

(21) Appl. No.: 19/231,999

(22) Filed: Jun. 9, 2025

Related U.S. Application Data
(63) Continuation-in-part of application No. 18/475,524, filed on Sep. 27, 2023, now Pat. No. 12,357,532.
(60) Provisional application No. 63/499,694, filed on May 2, 2023.

(51) Int. Cl. A63H 3/00; A61M 21/00; A63H 3/02

(52) U.S. Cl. CPC A63H 3/003; A61M 21/00; A63H 3/02; A61M 2021/0022

(57) ABSTRACT

A vibrotactile plush device can include at least one transducer configured to generate a plurality of vibrations, at least one control unit configured to receive an input signal from a communication protocol and to transmit a first output signal to the at least one transducer, and a power source. The device can include a vibrotactile material configured to transmit the plurality of vibrations at one or more restorative or therapeutic frequencies to a body of a user. A padding material can be configured to dampen the plurality of vibrations. In some embodiments, the vibrotactile plush device can also be a weighted device and further comprise a heating pad. In some embodiments, the plurality of vibrations can simulate the purring of a cat, and a cover can be configured to possess a cat shape.

### RELATED APPLICATIONS

This is a continuation-in-part application and so claims the benefit pursuant to 35 U.S.C. § 120 of a prior filed and co-pending U.S. non-provisional patent application Ser. No. 18/475,524, filed on Sep. 27, 2023, which itself claims priority pursuant to 35 U.S.C. § 119 (e) to and is entitled to the filing date of U.S. provisional patent application Ser. No. 63/499,694, filed on May 2, 2023. The contents of the aforementioned applications are incorporated herein by reference.

### BACKGROUND

The subject of this patent application relates generally to therapeutic plush devices, and more particularly to a vibrotactile cuddle device.

Applicant hereby incorporates herein by reference any and all patents and published patent applications cited or referred to in this application.

By way of background, vibrotactile components include transducers which can be provided within plush devices to deliver a vibratory effect on the user. Within media devices, the vibratory effect is intended to allow the user to feel the sound of the music. Within plush devices, the vibratory effect may be used for sensory stimulation. Weighted blankets and weighted toys can provide the user with a feeling of safety or security and can result in deep pressure stimulation. Heating pads and soft fabrics in plush devices can additionally provide somatosensory stimulation. However, a plush device, usable by children and adults, with multi-feature therapeutic elements like a substantive vibrotactile component, weights, a heating pad, and soft fabrics for providing a comprehensive soothing sensory device that further conveys possible healing features is not currently available. Accordingly, there remains a continuing need for a comprehensive soothing plush device.

Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a vibrotactile cuddle device. In at least one embodiment, In one aspect, a soothing device includes: a vibrotactile contact surface at a first side of the soothing device, the vibrotactile contact surface configured to be placed in contact with a body of a user; an outer shell comprising a padding cover surface separate from the vibrotactile contact surface; a vibrotactile material disposed at the vibrotactile contact surface; at least one transducer configured to couple vibrations into the vibrotactile material, wherein the vibrotactile material is configured to transmit the vibrations of the at least one transducer into the body of the user when the vibrotactile contact surface is in contact with the body of the user; a controller coupled to the at least one transducer, wherein the controller is configured to receive an input signal from a communication protocol and to transmit a first output signal to the at least one transducer, the controller further comprising a memory unit; at least one sensor for receiving external information to generate a response for adjusting an operation of the soothing device; a power source configured to provide power to the controller and the at least one transducer; and a padding material at least partially encapsulating the at least one transducer and the controller, wherein the padding material is configured to dampen the vibrations, wherein the padding material is positioned between the padding cover surface and the at least one transducer.

In some embodiments, the padding material comprises an acoustical foam. In some embodiments, the vibrations have a frequency of at least 20 Hz. In some embodiments, the controller is configured to transmit a second output signal to an audio device, wherein the second output signal is a wired or wireless signal. In some embodiments, the audio device comprises headphones, earbuds, or bone conduction headphones. In some embodiments, the second output signal comprises a sleep sound, a sleep message, a meditation sound, a meditation message, a story, a therapeutic sound, or a therapeutic message. In some embodiments the vibrotactile material comprises at least one of a microcellular elastomer, a polyurethane, a rubber, or a microcellular polyurethane. In some embodiments, the soothing device further comprises a heating pad disposed at least at the first side of the soothing device, proximate to the vibrotactile contact surface. In some embodiments the soothing device further comprises weights, configured to simulate a weight of a cat, a weighted cuddle toy, or a weighted blanket. In some embodiments, the power source comprises a rechargeable battery configured to be charged through conductive or inductive charging. In some embodiments, the soothing device further comprises a cover configured to have a shape of a cushion, a pad, a cat, a cuddle toy, or a vehicle. In some embodiments, the input signal is configured to adjust at least one of a power state, a volume, a run duration, a vibration intensity, or a vibration frequency. In some embodiments, the input signal comprises a user input communicated via a communication protocol to customize a vibration pattern generated by the at least one transducer. In some embodiments, the customization of the vibration pattern generated by the at least one transducer comprises decreasing a vibration frequency to deepen a perceived sound. In some embodiments, the memory unit is configured to store a library of sounds. In some embodiments, the soothing device further comprises a sound barrier material at least partially encapsulating the at least one transducer, wherein the sound barrier material is configured to dampen the vibrations, and wherein the sound barrier material is positioned between the padding material and the at least one transducer.

In another aspect, a vibrotactile plush device can include: a padding material at least partially surrounding a first region and a second region, wherein the first region is at a first side of the vibrotactile plush device and the second region is at a second side of the vibrotactile plush device, and wherein a cover at least partially encloses the padding material; at least one vibrotactile element comprising a transducer and a vibrotactile material, wherein the at least one vibrotactile element is disposed at the first region, and wherein the vibrotactile material is configured to transmit a plurality of vibrations of the transducer at one or more restorative frequencies to a body of a user, the vibrotactile material disposed between the transducer and the body of the user; a control unit coupled to the at least one vibrotactile element and disposed at the second region, wherein the control unit is configured to receive a plurality of input signals and to transmit a plurality of output signals; and a power source configured to provide power to the control unit and the transducer.

In some embodiments the padding material comprises an acoustical foam. In some embodiments, the padding material is not included between the transducer and a portion of an outer shell of the vibrotactile plush device, the portion of the outer shell configured to be placed into contact with the body of the user and to transmit the plurality of vibrations to the body of the user. In some embodiments, the plurality of vibrations has a restorative frequency value of at least 20 Hz. In some embodiments, the plurality of vibrations has a restorative frequency value between 20 Hz and 150 Hz. In some embodiments, the plurality of output signals further comprises a first output signal, and wherein the controller is configured to transmit the first output signal to an audio device, the first output signal comprising a wired or wireless signal. In some embodiments, the audio device comprises headphones, earbuds, or bone conduction headphones. In some embodiments, the first output signal comprises a sleep sound, a sleep message, a meditation sound, a meditation message, a story, a therapeutic sound, or a therapeutic message. In some embodiments, the vibrotactile material comprises at least one of a microcellular elastomer, a polyurethane, a rubber, or a microcellular polyurethane. In some embodiments, the vibrotactile plush device further comprises a heating pad disposed at least at the first side of the vibrotactile plush device proximate to the body of the user. In some embodiments, the vibrotactile plush device further comprises weights configured to simulate a weight of a cat, a weighted cuddle toy, or a weighted blanket. In some embodiments, the power source comprises a rechargeable battery configured to be charged through conductive or inductive charging. In some embodiments, the cover is configured to have a shape of a cushion, a pad, a cat, a cuddle toy, or a vehicle. In some embodiments, the plurality of input signals is configured to adjust at least one of a power state, a volume, a run duration, a vibration intensity, or a vibration frequency. In some embodiments, the plurality of input signals comprises a user input communicated via a communication protocol to customize a vibration pattern generated by the transducer. In some embodiments, customization of the vibration pattern generated by the at least one transducer comprises decreasing a vibration frequency to deepen a perceived sound. In some embodiments, the vibrotactile plush device further comprises a memory unit, wherein the memory unit is configured to store a library of sounds. In some embodiments, the vibrotactile plush device further comprises a sound barrier material at least partially encapsulating the at least one vibrotactile element, wherein the sound barrier material is configured to dampen the plurality of vibrations, and wherein the sound barrier material is positioned between the padding material and the at least one vibrotactile element. In some embodiments, the vibrotactile plush device further comprises at least one sensor for receiving external information to generate a response for adjusting an operation of the vibrotactile plush device.

In another aspect, a vibrotactile device comprises a housing, the housing further comprising at least one transducer configured to generate a plurality of vibrations, at least one control unit configured to receive an input signal from a communication protocol and to transmit a first output signal to the at least one transducer, and a power source; a vibrotactile material disposed at least partially on a surface of the housing, wherein the vibrotactile material is configured to transmit the plurality of vibrations at one or more restorative frequencies to a body of a user; and a padding material at least partially encapsulating the housing, wherein the padding material is configured to dampen the plurality of vibrations.

In some embodiments, the padding material is not included between the housing and a portion of an outer shell of the vibrotactile device, the portion of the outer shell configured to be placed into contact with the body of the user and to transmit the plurality of vibrations to the body of the user. In some embodiments, the plurality of vibrations has a restorative frequency value of at least 20 Hz. In some embodiments, the at least one control unit is configured to transmit a second output signal to an audio device, wherein the second output signal is a wired or wireless signal. In some embodiments, the audio device comprises bone conduction headphones. In some embodiments, the second output signal comprises at least one of a sleep sound, a sleep message, a meditation sound, a meditation message, a story, a therapeutic sound, or a therapeutic message. In some embodiments, the vibrotactile material comprises at least one of a microcellular elastomer, a polyurethane, a rubber, or a microcellular polyurethane. In some embodiments, the power source comprises a rechargeable battery configured to be charged through conductive or inductive charging. In some embodiments, the input signal is configured to adjust at least one of a power state, a volume, a run duration, a vibration intensity, or a vibration frequency. In some embodiments, the input signal comprises a user input communicated via a communication protocol to customize a vibration pattern generated by the at least one transducer. In some embodiments, the customization of the vibration pattern generated by the at least one transducer comprises decreasing a vibration frequency to deepen a perceived sound. In some embodiments, the at least one control unit further comprises a memory unit, wherein the memory unit is configured to store a library of sounds. In some embodiments, the vibrotactile device further comprises a sound barrier material at least partially encapsulating the housing, wherein the sound barrier material is configured to dampen the plurality of vibrations, wherein the sound barrier material is positioned between the padding material and the housing, and wherein the sound barrier material is not included between the surface of the housing and the vibrotactile material. In some embodiments, the vibrotactile device further comprises at least one sensor for receiving external information to generate a response for adjusting an operation of the vibrotactile device.

In another aspect, a method of operating a vibrotactile plush device can include: providing power to at least one controller and at least one vibrating transducer within the vibrotactile plush device; sending a first signal from a communication protocol to the at least one controller, wherein the first signal comprises instructions for operating the at least one vibrating transducer; and transmitting a plurality of vibrations from the at least one vibrating transducer through at least a vibrotactile material disposed between the at least one transducer and a body of a user according to the first signal.

In some embodiments, sending the first signal can further comprise controlling an intensity and a frequency of the plurality of vibrations. In some embodiments, sending the first signal can further comprise controlling a run duration of the plurality of vibrations. In some embodiments, sending the first signal further comprises sending a user input communicated via a communication protocol to customize a vibration pattern generated by the vibrating transducer.

In another aspect, a method of forming a vibrotactile plush device can include: providing an outer shell, wherein the outer shell comprises a padding cover surface and a vibrotactile contact surface; placing the vibrotactile contact surface at a first side of the vibrotactile plush device, wherein the vibrotactile contact surface is configured to be placed in contact with a body of a user; providing a vibrotactile material at the vibrotactile contact surface; providing at least one transducer configured to couple vibrations into the vibrotactile material, wherein the vibrotactile material is configured to transmit the vibrations of the at least one transducer into the body of the user when the vibrotactile contact surface is in contact with the body of the user; coupling a controller to the at least one transducer, wherein the controller is configured to receive an input signal from a communication protocol and to transmit a first output signal to the at least one transducer; coupling a memory unit to the controller; providing a power source configured to provide power to the controller and the at least one transducer; and providing a padding material at least partially encapsulating the at least one transducer and the controller, wherein the padding material is configured to dampen the vibrations, and wherein providing the padding material comprises positioning the padding material between the padding cover surface and the at least one transducer.

In some embodiments, the method further comprises providing at least one sensor for receiving external information for adjusting an operation of the vibrotactile plush device. In some embodiments, the input signal comprises a user input communicated via a communication protocol to customize a vibration pattern generated by the at least one transducer, and wherein the input signal is configured to adjust at least one of a power state, a volume, a run duration, a vibration intensity, or a vibration frequency.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1A is a schematic side sectional view of an exemplary vibrotactile plush device, according to one embodiment;

FIG. 1B is a schematic front sectional view of an exemplary vibrotactile plush device, according to one embodiment;

FIG. 1C is a schematic bottom sectional view of an exemplary vibrotactile plush device, according to one embodiment;

FIG. 2 is a schematic side sectional view of an exemplary vibrotactile element, according to one embodiment;

FIG. 3 is a schematic block diagram depicting an illustrative general architecture of an exemplary control unit;

FIG. 4 is a schematic side sectional view of a further exemplary vibrotactile plush device, according to one embodiment;

FIG. 5 is an example method of operating an exemplary vibrotactile plush device, according to one embodiment;

FIG. 6 is an example method of forming an exemplary vibrotactile plush device, according to one embodiment;

FIG. 7 is a schematic block diagram depicting a further illustrative general architecture of an exemplary control unit; and

FIG. 8 is a schematic side sectional view of a still further exemplary vibrotactile plush device, according to one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

Scientific research indicates that low-frequency vibrations may have therapeutic or healing effects. In one study on bone repair after injury, beneficial frequencies were determined to be from about 25 Hz to about 50 Hz. These and other frequencies (also referred to as restorative frequencies or restorative frequency values herein) falling in a range of about 25 Hz to about 150 Hz are used in pain relief and treatment of dyspnea, wounds, and more. This range of frequencies also corresponds to the frequencies of cat purrs. In addition to the possible physical therapies associated with cat purrs, cat purrs are also associated with calming effects in humans and can aid in stress relief. Sound resonation, generally, has been shown to viscerally stimulate the parasympathetic nervous system, decreasing stress and anxiety in recipients of this sound resonation.

In other scientific research, weighted cuddle toys and weighted blankets can provide sensory stimulation (e.g., deep pressure stimulation), facilitating the release of oxytocin, a neurotransmitter associated with positive well-being and anti-stress. Additionally, evidence shows that meditation can reduce psychological stress, with one study finding moderate evidence of improved anxiety, depression, and pain in response to meditation programs. Heat therapy is also known to provide human benefits, both physical and psychological.

Therefore, a plush device or cuddle toy comprising at least a vibrotactile component capable of transmitting vibrations at restorative frequencies, a comforting plush interior to hold onto while receiving the vibrations, and a calming or attractive exterior cover would greatly benefit both human children and adults. Such a cuddle toy further including weights and a heating pad would elevate the utility and functionality of the device, providing a unique and powerful fusion of human benefits.

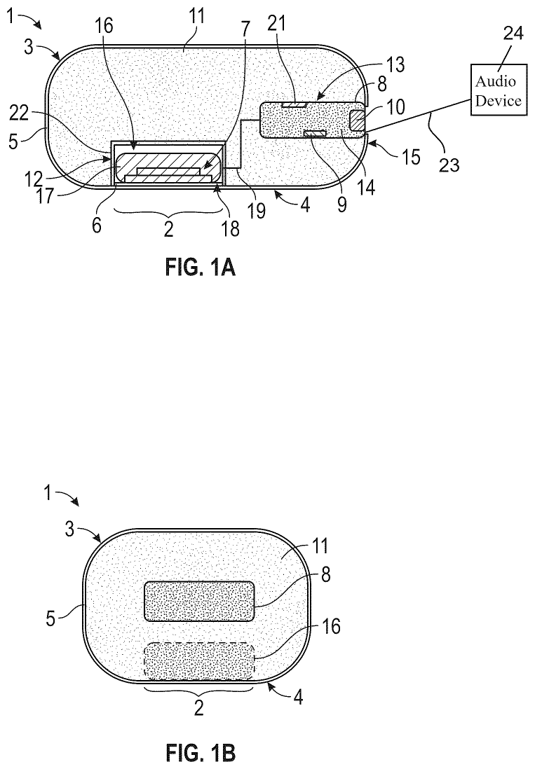

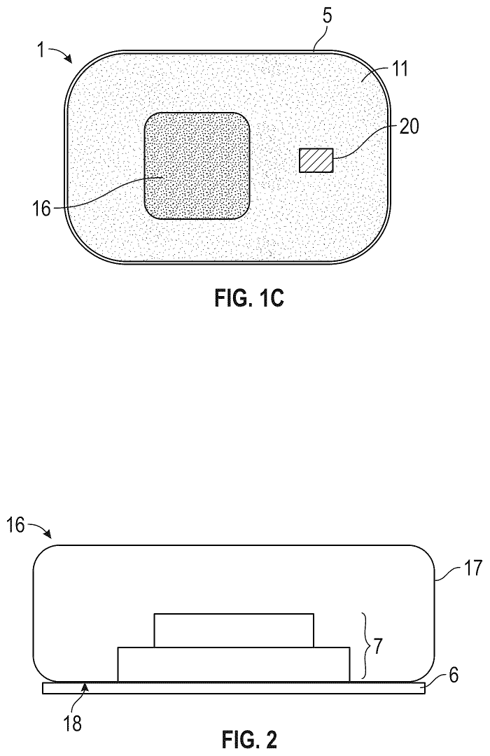

Various embodiments disclosed herein relate to vibrotactile plush devices, which may sometimes be referred to as “soothing devices” or “vibrotactile devices.” FIG. 1A illustrates a side schematic cross-sectional view of a vibrotactile plush device 1, according to one embodiment. In FIG. 1A, the vibrotactile plush device 1 comprises a padding material 11, a sound barrier material 22, a first region 12 at a first side 4 of the vibrotactile plush device 1 and a second region 13 at a second side 15 of the vibrotactile plush device 1, such that the padding material 11 at least partially surrounds the first and second regions, 12, 13.

In the configuration shown in FIG. 1A, a vibrotactile element 16 is disposed at the first region 12, comprising a transducer 7 and a vibrotactile material 6. The transducer 7 can be a vibrating mechanical actuator or an electroacoustic transducer, or exciter, or tactile transducer, or speaker, or subwoofer (hereinafter collectively referred to herein as an “electroacoustic transducer” or “transducer” for simplicity purposes), in which sound can be created when a vibrating transducer 7 is in direct contact with a rigid surface. For example, the transducer 7 can be a bone conducting-type transducer. In one embodiment, the transducer 7 can vibrate at a frequency (e.g., a restorative frequency value) of at least 20 Hz. In one embodiment, the transducer 7 can vibrate at a frequency of 25 Hz. In another embodiment the transducer 7 can vibrate at a frequency within a range of 20 Hz and 150 Hz. Still, in other embodiments, the transducer 7 can vibrate at a frequency greater than 150 Hz. In some embodiments, the transducer can have a frequency range of approximately 20 Hz to approximately 10,500 Hz. The transducer 7 can have an impedance of approximately 4 ohms and have an input power of 5 W. In some embodiments, the transducer 7 can have an input power value ranging between approximately 3 W and approximately 10 W. In some embodiments the impedance can range from approximately 4 ohms to approximately 8 ohms. The transducer 7 can have a sound pressure level of approximately 88 dB, and it can have a sensitivity rating of approximately 85 dB. In some embodiments, the sensitivity rating can be greater than 85 dB. For example, the sensitivity rating can be 90 dB. The transducer 7 can have an amplitude of oscillation of approximately 1 mm, and in some embodiments, the amplitude of oscillation can be between approximately 1 mm and approximately 2 mm. The transducer 7 can have a directivity factor of 1.8. In some embodiments, the directivity factor value can range from approximately 1.5 to approximately 2.

An advantage is that a user can possibly experience health benefits from the vibrotactile plush device 1 imparting such frequencies to the user's body. Such possible health benefits can include improved mental wellbeing and physical benefits such as improved bone health. Although the vibrotactile device 1 of FIG. 1A includes one transducer 7, in other embodiments, the vibrotactile device 1 can comprise multiple transducers.

In the configuration shown in FIG. 1A, padding material 11 is not included between the transducer 7 and the vibrotactile material 6, and the padding material 11 is not included between the vibrotactile material 6 and a vibrotactile contact surface 2. The vibrotactile contact surface 2 is a portion of an outer shell 5 that encapsulates the device 1. The outer shell 5 further comprises a padding cover surface 3, such that the padding cover surface 3 and vibrotactile contact surface 2 are different portions of the outer shell 5. The padding cover surface 3 is disposed over the padding material 11 (i.e., the cover at least partially encloses or encases the padding material 11). The vibrotactile contact surface 2 is disposed at the first side 4 of the vibrotactile plush device 1, and the vibrotactile material 6 is disposed at the vibrotactile contact surface 2. A user (e.g., a child at least 6 months old or an adult) can place the vibrotactile contact surface 2 of the vibrotactile plush device 1 in direct contact with the user's body (e.g., a user's upper body). For example, the user can place the vibrotactile contact surface 2 of the device 1 proximate to the user's chest (e.g., sternum) or abdomen. In this configuration, the padding material provides a user with the ability to hold onto the vibrotactile plush device 1 comfortably, and the placement of the padding material does not impede the delivery of the vibrations from the transducer 7 through the vibrotactile material 6 disposed at the vibrotactile contact surface 2. In at least one further embodiment, the vibrotactile plush device 1 provides an engagement mechanism configured for maintaining the vibrotactile plush device 1 in contact with the user's body in a “hands free” manner. In at least one such embodiment, the engagement mechanism is configured as a garment, such as an oversized t-shirt, for example. In at least one alternate such embodiment, the engagement mechanism is configured as a sleeve, a sling, or an elastic band capable of being removably engaged with the user's body, such that the vibrotactile plush device 1 is sandwiched therebetween. In still further such embodiments, the engagement mechanism may take on any other sizes, shapes and/or configurations, now known or later developed, so long as the engagement mechanism is capable of maintaining the vibrotactile plush device 1 in contact with the user's body.

The vibrotactile material 6 is a thin layer of material that transmits the vibrations of the transducer 7. For example, the vibrotactile material 6 can have a thickness of approximately ⅛ of an inch. In other examples, the vibrotactile material 6 can have a thickness from ⅛ of an inch to ¼ of an inch. In some embodiments, the thickness of the vibrotactile material 6 can be less than ⅛ of an inch or greater than ¼ of an inch. The vibrotactile material 6 can be a microcellular elastomer, a polyurethane, a rubber, a microcellular polyurethane, or other such material suitable for softening the surface through which the transducer vibrations are delivered and for transmitting the vibrations to a user's body. In some embodiments, the vibrotactile material 6 comprises one of the materials described above. In other embodiments, the vibrotactile material 6 can be a combination of two or more of the materials described above. In some embodiments, the vibrotactile material 6 is neoprene.

The padding material 11 can be a foam, such as an acoustical foam. In another embodiment, the padding material 11 can be a memory foam, providing comfort to the user and warming the user, owing to the denseness of the material, which traps body heat. In other embodiments, the padding material 11 can include at least one of a foam, a padding, or a fabric of substantial thickness similar to what would be present in a cuddle toy. The padding material 11 dampens the vibrations or sound emitted from the transducer 7 such that the effects of the transducer 7 are focused on the user and are non-disruptive or minimally disruptive to others who are nearby the user. Because of the dampening effects of the padding material 11, others who are nearby the user cannot hear the vibrations or sound from the vibrotactile device 1 or may hear the vibrations or sound at a very low volume. In some embodiments, the padding material 11 can enhance the vibrations or sound emitted from the transducer 7. For example, a firm or stiff foam like HR70 can be included. In some embodiments, the padding material 11 can include a mixture of firm and soft padding material or foam, such that the firm padding material can enhance the vibrations and the soft padding material can dampen the vibrations or sound emitted from the transducer 7 while providing a softness to the plush device.

The padding material 11 can have a range of thicknesses. For example, the padding material 11 can have thicknesses ranging from approximately 0.5 inches to approximately 3.5 inches. In some embodiments, the padding material 11 can be a single piece of padding material with regions carved out for placement of the vibrotactile element 16 and the control unit 8. In some embodiments, the padding material 11 can be a plurality of padding material pieces or layers coupled together. For example, in some embodiments, the padding material 11 that comprises the bottom surface 4 of the vibrotactile plush device 1 outside of the vibrotactile contact surface 2, can have a thickness ranging from approximately 0.5 inches to approximately 1.25 inches. The padding material comprising the top surface of the device (e.g., the surface opposite the bottom surface 4) can have a thickness ranging from approximately 0.5 inches to approximately 3.5 inches.

The sound barrier material 22 is disposed between the vibrotactile element 16 and the padding material 11, and the sound barrier material 22 is not included between the vibrotactile element 16 and the vibrotactile contact surface 2. The sound barrier material 22 dampens the vibrations or sound emitted from the transducer 7. In some embodiments the sound barrier material 22 comprises mass loaded vinyl (MLV). In some embodiments the sound barrier material 22 has a thickness of approximately ¼ of an inch. In other embodiments, the sound barrier material 22 can have a thickness greater than ¼ of an inch or less than ¼ of an inch. The sound barrier material 22 is configured to act as an additional dampening layer, minimizing the vibrations or sound from the vibrotactile plush device 1 from reaching non-users in the vicinity of the user. In some embodiments, the sound barrier material 22 is disposed between the vibrotactile element 16 and the padding material 11 on all sides of the vibrotactile element 16 except for the side of the vibrotactile element 16 proximate and parallel to the vibrotactile contact surface 2 (as shown for example in FIG. 1A). In some embodiments, the sound barrier material 22 is disposed on one side of the vibrotactile element 16, wherein the one side is opposite the side of the vibrotactile element 16 proximate and parallel to the vibrotactile contact surface 2.

In some embodiments, the cover can take the shape of a cat. An advantage of such a cover on the vibrotactile plush device 1 is that users who are allergic to cats or live in households with family members or other residents presenting with cat allergies can enjoy the health benefits and companionship of a device that can resemble a cat and output a therapeutic, cat-like purr. In some embodiments, the cover can take the shape of a cushion, a pad, a cat, a cuddle toy, or a vehicle (e.g., a car, plane, train, boat, etc.). In one embodiment, the cover is the outer shell 5 of the device 1. In another embodiment, the cover is separate from and disposed over the outer shell 5 of the device 1, which enables the user to remove the cover for cleaning or sanitization. In at least one alternate embodiment, the cover may be omitted completely.

A control unit 8 is disposed at the second region 13, and it comprises a controller 14, a power source 10, and a memory unit 9. In one embodiment the power source 10 provides power to the vibrotactile plush device 1. The vibrotactile plush device 1 can include a power source comprising a rechargeable battery, which can be charged through conductive or inductive charging. In one example, the device 1 can be charged or recharged inductively by setting the device 1 onto a battery charging plate, allowing for automatic charging. In another embodiment, the device 1 can be charged through conductive charging, which can include charging the device through a USB connection or power jack. In at least one embodiment, the control unit 8 provides an at least one indicator light positioned and configured for assisting the user with operating the control unit 8 in low light environments. The memory unit 9 is configured to store information pertinent to operating the device 1. For example, the memory unit 9 can store a library of sounds to be played on the device 1. In another example, the memory unit 9 can retain information regarding the sound and run duration from one use to another use, unless the user changes the settings. Thus, the user does not have to reset the vibrotactile plush device 1 to their desired settings each time the device 1 is to be used. In some embodiments, the control unit comprises a tone or bass controller that facilitates an increase or decrease in the amount of bass in the sound. In such embodiments, the tone or base controller allows for adjusting the vibrations.

In the configuration in FIG. 1A, the control unit 8 and the vibrotactile element 16 are electrically connected through a wire 19, facilitating transmission of a first output signal to the vibrotactile element 16. In some embodiments, the control unit 8 can also include a physical on/off switch for turning the vibrotactile device 1 on or off. In some embodiments, the user can adjust the volume of the vibrotactile device 1 through a physical volume element disposed at a surface of the control unit 8 external to the device 1. The first output signal instructs the transducer 7 to transmit vibrations of a specific frequency and amplitude. In one embodiment, the controller 14 transmits a plurality of output signals. A communication protocol provides input signals to the control unit 8. The communication protocol allows a user to link or connect their vibrotactile plush device 1 to other electronic devices such as cellular phones, tablets, computers (including laptops and desktops), and the like.

In one embodiment, the controller 14 transmits a second output signal that can be a wired or wireless signal 23 to an audio device 24, such as headphones, earbuds, or bone conduction headphones. The second output signal can comprise a variety of sounds or messages. For example, the second output signal can be a sound that helps soothe one to sleep (e.g., a sleep sound). In another example, the second output signal can be a meditation sound, suitable for meditating. In another example, the second output signal can be a therapeutic sound, imparting calming or soothing sounds to the user of the vibrotactile device 1. In another example, the second output signal can comprise messages, such as sleep messages, meditation messages, or therapeutic messages, wherein each is intended to produce a soothing or calming effect on the user. In another example, the second output signal can be a story. For instance, the story may be a pre-recorded story in a parent's voice that can be played to a child through the device.

In some embodiments, the vibrotactile device 1 can further comprise one or more sensors 20, 21 for receiving external information to generate a response for adjusting an operation of the vibrotactile device 1. The external information can include an orientation or a position of the vibrotactile device 1, a breathing pattern or a breathing rate of the user, and more. Adjusting an operation of the vibrotactile device 1 can include adjusting at least one of a power state (e.g., turning the device on or off), a volume, a run duration, a vibration intensity, a vibration frequency, etc. The one or more sensors 20, 21 can be configured to receive/sense the external information and to send a signal to the control unit 8 to turn the vibrotactile device 1 on or off in response to the external information. For example, a first sensor 21 can be configured to detect if the vibrotactile device 1 has fallen off a sleeping child or adult (i.e., receive external information) and transmit a signal to the control unit 8 to turn the vibrotactile device 1 off. In some embodiments, the first sensor 21 is a gyroscope sensor disposed in the control unit 8 and senses device position. In another example, a second sensor (not shown) can be configured to detect if the user is crying and to transmit a signal to the control unit 8 to turn the vibrotactile device 1 on. In another example, and as shown in FIG. 1C, a third sensor 20 can be disposed at the bottom of the vibrotactile device 1. In some embodiments, this third sensor 20 can be a piezoelectric sensor configured to detect a user's breathing pattern, enabling the vibrotactile device 1 to produce a sound output that matches the breathing pattern detected by the third sensor 20. For example, a sound or plurality of sounds can be loaded into the device 1 and software can be configured to adjust the sound to match the breathing pattern detected by the third sensor 20. In some examples, this sound output can be a cat purr; however, in further embodiments, as discussed below, the sound output may be any other type of sound, now known or later developed, such as an idling motorcycle engine, a drum, meditation sounds, a person's voice, etc. As such, in at least one embodiment, the sound output may be a friend or family member's voice (such as a father's voice telling a children's bedtime story, for example), so as to help soothe the user. Additionally, in at least one embodiment, the vibrotactile device 1 is configured for producing one sound in sync with the user inhaling, and producing a different sound in sync with the user exhaling, thereby providing alternating inhalation and exhalation segments of sounds and/or vibrations at a predetermined frequency. In at least one embodiment, the vibrotactile device 1 is configured for producing a sound that gradually slows so as to cause the user to likewise slow their own breathing. In at least one further embodiment, the vibrotactile device 1 is configured for facilitating box breathing exercises with the user—e.g., inhaling, holding, exhaling, and holding again, typically for four counts each.

In the illustrated embodiment of FIGS. 1A and 1C, the vibrotactile device 1 can comprise at least one sensor 20, 21. The inclusion of at least one sensor 20, 21 makes possible the creation of a feedback system that enables the vibrotactile device 1 to operate in various manners depending on the inputs received from the at least one sensor 20, 21. As described above, the vibrotactile device 1 can include a variety of sensors 20, 21 that enable the vibrotactile device 1 to detect various inputs, which reduces the need for constant non-user supervision or intervention to make sure the vibrotactile device 1 has been turned on or off depending on the user's state and which helps reduce the device's power consumption during inactive phases (e.g., when a child has fallen asleep). Although the vibrotactile device 1 of FIGS. 1A and 1C illustrate the inclusion of two sensors 20, 21, more or fewer sensors can be included.

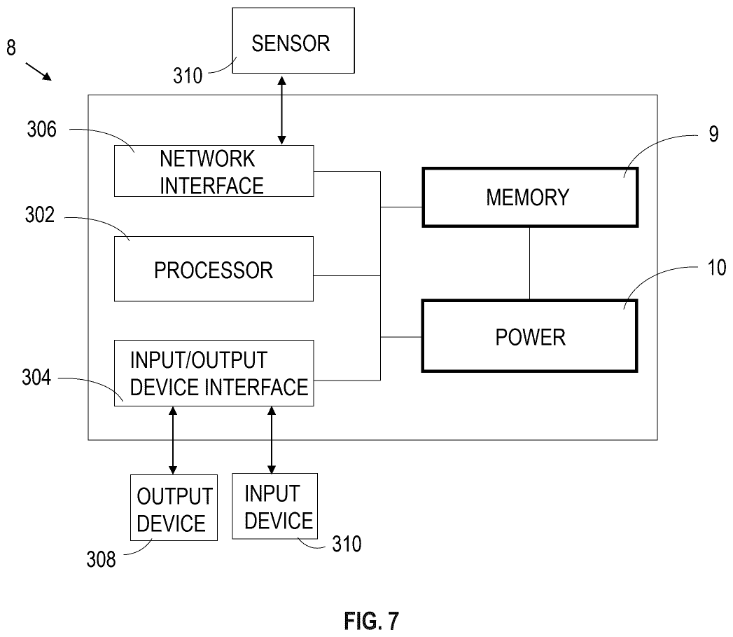

In at least one further embodiment, as illustrated in FIG. 7, at least one sensor 20 may be positioned external to and remote from the vibrotactile device 1 while being in wireless communication with a network interface 306 of the control unit 8 of the vibrotactile device 1 (discussed in detail below). In at least one such embodiment, the at least one external sensor 20 is configured as a wearable device, such as a watch, a bracelet or an arm band, for example. In at least one embodiment, the at least one external sensor 20 is built into a third-party wearable device, such as a smartwatch or a fitness tracker, for example. In at least one such embodiment, the at least one external sensor 20 is configured for tracking the user's breathing and/or heartrate, with the control unit 8 configured for wirelessly receiving that sensor data and dynamically adjusting one or more of the sound, vibration intensity or vibration frequency produced by the vibrotactile device 1 to match the breathing pattern and/or heartrate detected by the at least one external sensor 20. With the at least one external sensor 20 engaged with the user, the control unit 8 of the vibrotactile device 1 is able to more consistently detect the user's breathing and/or heartrate, regardless of the specific position and/or orientation of the vibrotactile device 1 relative to the user's body. In at least one embodiment, the vibrotactile device 1 provides no onboard or internal sensors, and instead only utilizes the at least one external sensor 20.

In some embodiments, a website comprising a library of sounds can be used in combination with the vibrotactile device 1. A community of users utilizing this website can communicate and compare their experiences with the vibrotactile device, which can help improve the experiences for other users of the device. The website can additionally facilitate uploading sounds, messages, and stories such that other users of the website and device can partake in these shared sounds, messages, and stories. In some embodiments, the sounds, messages, and stories are user-created. The library of sounds can be downloaded to the vibrotactile device 1. In one embodiment, the library of sounds comprises sound alternatives. For example, a user can take a sound or message (e.g., a pre-existing or newly recorded custom personal message, mantra, or story) and lower its frequency, to deepen a perceived sound or message (e.g., decreasing the vibration frequency of the vibrational pattern), which may increase soothing qualities when transmitted or played back through the vibrotactile device 1.

In some embodiments, the communication protocol is a part of a mobile application that facilitates comprehensive remote or wireless control of the sounds. The mobile application can include a variety of user controls to be operated with the device. For example, from the mobile application in one embodiment, the user could control the power state (e.g., turn on/off) of the vibrotactile plush device 1, the volume, the time played, the sequence of sounds played, loading sounds (including a library of sounds) onto the vibrotactile device 1, the vibration or percussion intensity, the vibration frequency, streaming sounds with Bluetooth® and/or Wi-Fi, and setting parental controls (e.g., a volume limit and vibration intensity limit for babies and children).

Although FIG. 1A illustrates the first side 4 being different from the second side 15, in some embodiments the first side 4 can be the same as the second side 15 of the vibrotactile plush device 1. Moreover, the vibrotactile plush device 1 is a portable device that can comprise a variety of sizes or dimensions. In one embodiment, the vibrotactile device 1 has the dimensions of 6 inches wide by 9 inches long by 3.5 inches in height. In another embodiment, the vibrotactile plush device 1 has the dimensions of 8 inches wide by 11 inches in length by 5 inches in height. In some embodiments, the vibrotactile plush device 1 can have a width between approximately 4 inches and approximately 8 inches; a length between approximately 5 inches and approximately 11 inches; and a height between approximately 2.5 inches and approximately 5 inches. In some embodiments, the vibrotactile plush device 1 has the dimensions of 4 inches wide by 5 inches long by 2.5 inches in height. In some embodiments, the vibrotactile plush device 1 has the dimensions of 8 inches wide by 11 inches long by 5 inches high. In some embodiments, the vibrotactile plush device 1 can weigh approximately 11 oz. In some embodiments, the vibrotactile plush device 1 can weigh approximately 2 lbs or weigh between approximately 2 lbs and 3 lbs. In some embodiments, the vibrotactile plush device 1 can have a weight between approximately 11 oz. and 3 lbs.

The size or dimension of the vibrotactile plush device 1 can vary so as to correspond to users of varying sizes (e.g., a child or an adult), improving the soothing or calming effect of the vibrotactile device 1 for users of different sizes. For example, the vibrotactile device can lend itself to cuddling because of its size. Further, because of the size and portability of the vibrotactile plush device 1, the device can be used in numerous circumstances and readily stored when not in use. For example, the user can facilely transport it to an office location or to a place of relaxation (e.g., a space or room where one could relax and simultaneously carry out other activities such as watching television). Additionally, the user can store the device on a bedside table or in a storage container/cabinet or place it on a bed, where the vibrotactile plush device 1 has a relatively small footprint.

In some embodiments, the vibrotactile plush device 1 comprises a heating pad disposed at least at the first side 4 of the soothing device 1. The heating pad facilitates warming the foam base of the vibrotactile plush device 1. In one embodiment, the warmth of the heating pad simulates the warmth of a cat.

In some embodiments, the vibrotactile plush device 1 comprises weights. In one embodiment, the weights can be disposed at least at the first side 4 of the vibrotactile plush device 1. The weights can comprise beads (e.g., micro glass beads or steel beads), grains, plastic poly pellets, sand, pebbles, etc. In one embodiment, the weights comprise a weight simulating that of a cat, a weighted cuddle toy, or a weighted blanket. In some embodiments, the weights can vary between 1 pound and 5 pounds. In other embodiments, the weights can weigh between 1 pound and 10 pounds. In some embodiments the weights can be customized and weigh up to 10% of the user's weight. A weighted vibrotactile plush device 1 can replicate the secure feeling a user could receive with a weighted blanket but have the smaller and more portable feature of a cuddle toy.

FIGS. 1B-C illustrate a front cross-sectional view and a bottom cross-sectional view of the vibrotactile plush device 1 of FIG. 1A, respectively. In FIG. 1B the control unit 8 is positioned in a plane above that of the vibrotactile element 16. The vibrotactile element 16 is illustrated to be positioned deeper within the soothing device 1 as compared to the control unit 8, and the vibrotactile element 16 is disposed at the first side 4 of the soothing device 1, corresponding to the vibrotactile contact surface 2. In FIG. 1C, a bottom cross-sectional view of the vibrotactile element 16 is depicted. The vibrotactile material 6 is disposed at this surface such that vibrations generated by the transducer 7 are transmitted to the body of a user through the vibrotactile material 6.

FIG. 2 illustrates a schematic side cross-sectional view of the vibrotactile element 16 of FIG. 1A. In FIG. 2, the vibrotactile element 16 comprises a housing 17, the housing 17 further comprising the transducer 7. A vibrotactile material 6 is coupled to the surface of the housing 18. In some embodiments, the vibrotactile material 6 comprises a soft layer of foam, rubber, or latex. In some embodiments, the transducer is located in a housing or transducer box 17 comprising a rigid material (e.g., hard plastic material). In some embodiments, the vibrotactile material 6 can be disposed under the rigid material, which can dampen strong vibrations from the transducer 7 to a comfortable or tolerable amount on the user. In some embodiments, the vibrotactile material 6 can be disposed between a surface of the transducer box 17 and the vibrotactile contact surface 2. The housing 17 can be a hard plastic chassis. In some embodiments, the control unit 8 can be housed in a chassis comprising the same hard plastic material as that used in housing 17.

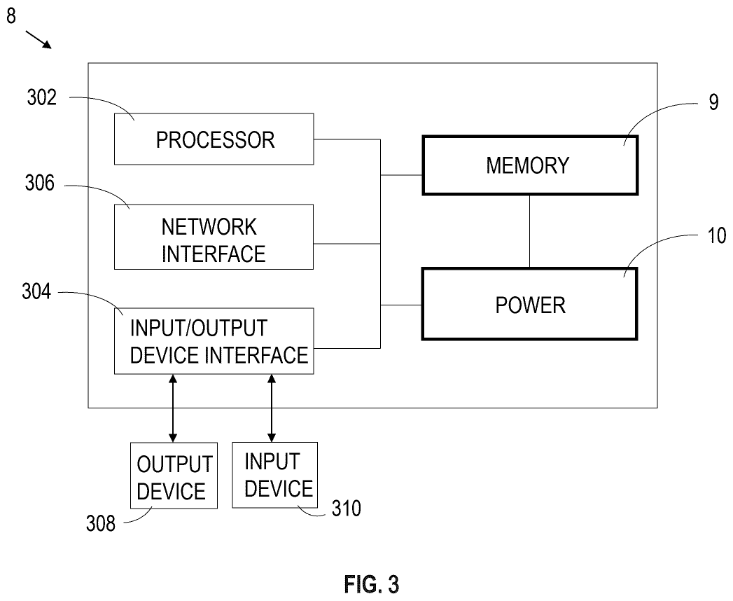

FIG. 3 illustrates a schematic block diagram depicting an illustrative general architecture of a control unit 8 for implementing aspects of the vibrotactile plush device 1. The control unit 8 can include more (or fewer) components than those shown in FIG. 3. The control unit 8 may include a processor 302 and an input/output device interface 304. In some embodiments, the input/output device interface 304 can include a panel for controlling the power; adjusting the volume, bass, and/or treble; switching between various sound files; and providing a power source (e.g., facilitating a micro-USB connection to allow for charging/recharging of the power source). In some embodiments, the control unit 8 may include a network interface 306. In some embodiments, network interface 306 can allow for short-range wireless connections (e.g., Bluetooth® connection). The control unit 8 components may communicate with one another by way of a communication bus. As illustrated, the control unit 8 is associated with, or in communication with, at least one output device 308 and at least one input device 310. For example, the output device 308 can be the electroacoustic transducer (or exciter) 7. Additionally, the control unit 8 may include one or more sensors 20, 21 (described herein) in electrical communication with the processor 302, which are capable of sensing external information that can be used by the processor 302 to regulate the operation of the vibrotactile plush device 1. In at least one alternate embodiment, where there is at least one external sensor 20 as discussed above, the at least one external sensor 20 is in wireless communication with the network interface 306 of the control unit 8 in order to provide sensor data to the control unit 8. The network interface 306 may provide the control unit 8 with connectivity to one or more networks or computing systems. The processor 302 can thus receive information and instructions from other control units or services via a network (e.g., wireless personal area network (WPAN)). The processor 302 may also communicate to and from the memory 9 and further provide output information (e.g., audio data) for an output device 308 (e.g., audio device 24 like headphones) via the input/output device interface 304. The input/output device interface 304 may accept input from the input device 310 (e.g., bass adjustment information). The memory 9 may contain specifically configured computer program instructions that can be executed by the processor 302. In some embodiments, the memory 9 may include RAM, ROM and/or other persistent or non-transitory computer-readable storage media.

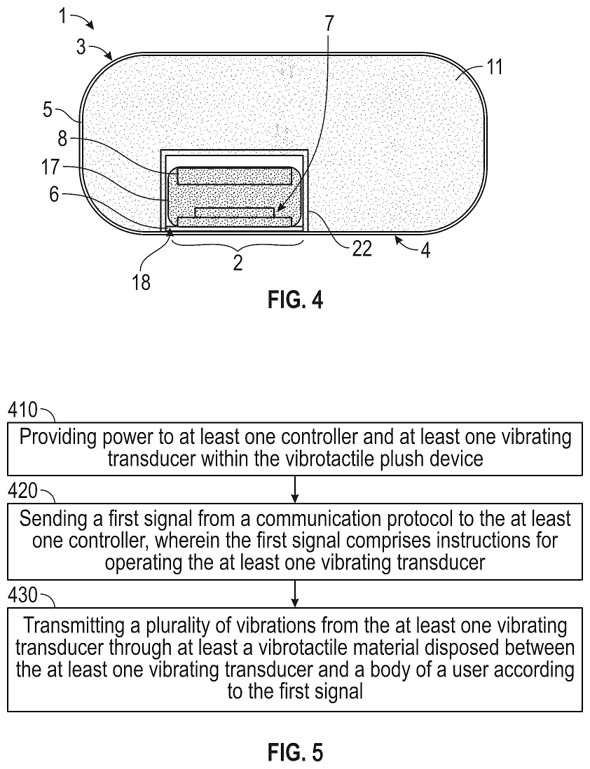

FIG. 4 illustrates another embodiment of the vibrotactile plush device 1 shown in FIG. 1A. Unless otherwise noted, the elements of FIG. 4 are the same as or generally similar to the elements of FIG. 1A, and alternatives noted above with respect to FIG. 1A are likewise applicable to the embodiment of FIG. 4. In FIG. 4, the vibrotactile plush device 1 comprises a padding material 11, a sound barrier material 22, and a housing 17, the housing 17 further comprising a transducer 7, a vibrotactile material 6 disposed at a surface of the housing 18, and a control unit 8. Unlike in FIG. 1A, in FIG. 4, the transducer 7 and control unit 8 can be combined in one housing 17, as shown. Consequently, no padding material 11 is disposed between the transducer 7 and control unit 8. In this configuration, the vibrotactile contact surface 2 is a portion of an outer shell 5 that encapsulates the soothing device 1. The outer shell 5 further comprises a padding cover surface 3, such that the padding cover surface 3 and vibrotactile contact surface 2 are different portions of the outer shell 5. The padding cover surface 3 is disposed over the padding material 11, the vibrotactile contact surface 2 is disposed at the first side 4 of the vibrotactile plush device 1, and the vibrotactile material 6 is disposed at the vibrotactile contact surface 2, corresponding to a surface of the housing 18. In some embodiments, the housing 17 is removable. In such an embodiment, the localization of the transducer 7 and the control unit 8 to a single location or region within the vibrotactile device 1 affords an advantage of maintaining the devices within a single housing within the plush device. When in a single housing, the entirety of the hardware system can be facilely removed from the plush device, improving the feasibility of cleaning or sanitizing the remaining portion of the plush device (e.g., placing the remaining portion of the plush device into a washing machine).

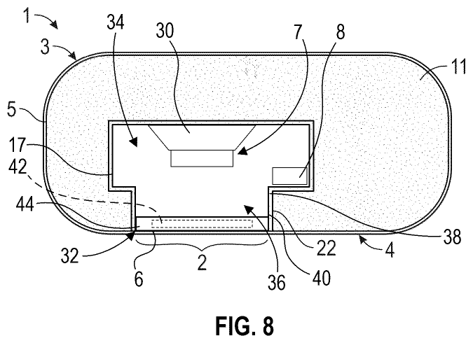

FIG. 8 illustrates yet another embodiment of the vibrotactile plush device 1 shown in FIG. 1A. Unless otherwise noted, the elements of FIG. 8 are the same as or generally similar to the elements of FIG. 1A, and alternatives noted above with respect to FIG. 1A are likewise applicable to the embodiment of FIG. 8. In FIG. 8, the vibrotactile plush device 1 comprises a padding material 11, a sound barrier material 22, and a housing 17, with a transducer 7 and a control unit 8 positioned within the housing 17. In at least one such embodiment, the housing 17 is an airtight enclosure, while the transducer 7 comprises an active speaker 30 (such as a loudspeaker or a subwoofer, for example) and a substantially planar passive radiator 32. In such embodiments, the active speaker 30 displaces air in the housing 17, which drives the passive radiator 32 to produce deeper bass sounds along with a stronger tactile feedback. In at least one embodiment, the housing 17 provides a main compartment 34, within which each of the active speaker 30 and control unit 8 are positioned, and a radiator tube 36 in fluid communication with the main compartment 34. In at least one embodiment, a first end 38 of the radiator tube 36 is engaged with the main compartment 34, while an opposing second end 40 of the radiator tube 36 is open-ended and is positioned proximal to but spaced apart from the bottom surface of the padding material 11 and a bottom edge of the sound barrier material 22. In at least one embodiment, the radiator tube 36 has an inner diameter of approximately 2 inches. However, in further embodiments, the inner diameter of the radiator tube 36 may be less than or greater than 2 inches. In at least one embodiment, the active speaker 30 is coupled to an interior surface of the side wall of the main compartment 34. Additionally, in at least one embodiment, the passive radiator 32 is engaged with the second end 40 of the radiator tube 36, with the passive radiator 32 functioning as the non-padded vibrotactile material 6, spanning the open second end 40 of the radiator tube 36 such that an exterior surface of the planar, non-padded vibrotactile material 6 lies in the same plane as the bottom surface of the padding material 11 and the bottom edge of the sound barrier material 22. In at least one embodiment, the passive radiator 32 provides a weighted disc 42 surrounded circumferentially by a diaphragm 44 constructed out of a relatively flexible, non-padded vibrotactile material, such as rubber, for example, with a perimeter edge of the diaphragm 44 engaged with the second end 40 of the radiator tube 36. In at least one further embodiment, the weighted disc 42 is embedded within or otherwise substantially encapsulated by the diaphragm 44. In still further embodiments, the passive radiator 32 may take on any other sizes, shapes, dimensions and/or configurations now known or later developed, so long as the passive radiator 32 is capable of substantially carrying out the functionality described herein. In such embodiments, the planar, non-padded vibrotactile material 6 (i.e., the diaphragm 44 of the passive radiator 32) cooperates with the bottom surface of the padding material 11 and the bottom edge of the sound barrier material 22 to form a continuous planar surface for being placed in contact with the body of the user. In at least one embodiment, the active speaker 30 is positioned on the side wall of the main compartment 34 so as to be substantially opposite the opening of the second end 40 of the radiator tube 36. However, in further embodiments, the active speaker 30 may be positioned elsewhere on the side wall of the main compartment 34, so long as the device 1 is substantially capable of carrying out the functionality described herein. Unlike in FIG. 1A, in FIG. 8, the transducer 7 and control unit 8 can be combined in one housing 17, as shown. Consequently, no padding material 11 is disposed between the transducer 7 and control unit 8. In this configuration, the vibrotactile contact surface 2 is a portion of an outer shell 5 that encapsulates the soothing device 1. The outer shell 5 further comprises a padding cover surface 3, such that the padding cover surface 3 and vibrotactile contact surface 2 are different portions of the outer shell 5. The padding cover surface 3 is disposed over the padding material 11, the vibrotactile contact surface 2 is disposed at the first side 4 of the vibrotactile plush device 1, and the vibrotactile material 6 is disposed at the vibrotactile contact surface 2. In some embodiments, the housing 17 is removable. In such an embodiment, the localization of the transducer 7 and the control unit 8 to a single location or region within the vibrotactile device 1 affords an advantage of maintaining the devices within a single housing 17 within the plush device 1. When in a single housing 17, the entirety of the hardware system can be facilely removed from the plush device 1, improving the feasibility of cleaning or sanitizing the remaining portion of the plush device 1 (e.g., placing the remaining portion of the plush device 1 into a washing machine). As noted above, in at least one embodiment, the device 1 includes an exterior cover; and in at least one alternate embodiment, the cover may be omitted completely.

FIG. 5 illustrates an example method of operating a vibrotactile plush device 1. Individual operations are shown in each block. Unless otherwise noted, vibrotactile plush device 1 elements included within the method of FIG. 5 are the same as or generally similar to the elements of FIGS. 1 through 4, and alternatives noted above with respect to the apparatus elements of FIGS. 1 through 4 are likewise applicable to the method disclosed in FIG. 5. Block 410 presents a step comprising providing power to at least one controller 14 and at least one vibrating transducer 7 within the vibrotactile plush device 1. Block 420 presents a step comprising sending a first signal from a communication protocol to the at least one controller 14, wherein the first signal comprises instructions for operating the at least one vibrating transducer 7. The first signal can comprise instructions for controlling an intensity, frequency, and/or run duration of the plurality of vibrations. The first signal can further comprise sending a user input communicated via a communication protocol to customize a vibration pattern generated by the at least one vibrating transducer 7. Block 430 presents a step comprising transmitting a plurality of vibrations from the at least one vibrating transducer 7 through at least a vibrotactile material 6 disposed between the at least one vibrating transducer 7 and a body of a user according to the first signal.

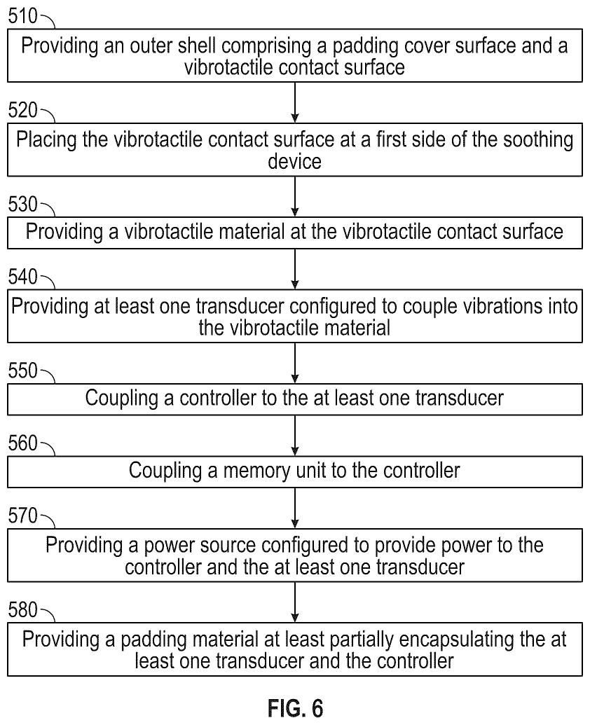

FIG. 6 illustrates an example method of forming a vibrotactile plush device 1. Individual operations are shown in each block. Unless otherwise noted, vibrotactile plush device 1 elements included within the method of FIG. 6 are the same as or generally similar to the elements of FIGS. 1 through 4, and alternatives noted above with respect to the apparatus elements of FIGS. 1 through 4 are likewise applicable to the method disclosed in FIG. 6. Block 510 presents a step comprising providing an outer shell 5, wherein the outer shell 5 comprises a padding cover surface 3 and a vibrotactile contact surface 2. Block 520 presents a step comprising placing the vibrotactile contact surface 2 at a first side 4 of the soothing device 1, wherein the vibrotactile contact surface 2 is configured to be placed in contact with a body of a user. Block 530 presents a step comprising providing a vibrotactile material 6 at the vibrotactile contact surface 2. Block 540 presents a step comprising providing at least one transducer 7 configured to couple vibrations into the vibrotactile material 6, wherein the vibrotactile material 6 is configured to transmit the vibrations of the at least one transducer 7 into the body of the user when the vibrotactile contact surface 2 is in contact with the body of the user. Block 550 presents a step comprising coupling a controller 14 to the at least one transducer 7, wherein the controller 14 is configured to receive an input signal from a communication protocol and to transmit a first output signal to the at least one transducer 7. In some embodiments, the input signal can include a user input communicated through the communication protocol to customize a vibration pattern to be generated by the at least one transducer 7. In some embodiments, the input signal can be configured to adjust a power state, a volume, a run duration, a vibration intensity, or a vibration frequency of the vibrotactile device 1. Block 560 presents a step comprising coupling a memory unit 9 to the controller 14. Block 570 presents a step comprising providing a power source 10 configured to provide power to the controller 14 and the at least one transducer 7. Block 580 presents a step comprising providing a padding material 11 at least partially encapsulating the at least one transducer 7 and the controller 14, wherein the padding material 11 is configured to dampen the vibrations, and wherein providing the padding material 11 comprises positioning the padding material 11 between the padding cover surface 3 and the at least one transducer 7.

In some embodiments, the method of forming a vibrotactile plush device 1 can include providing at least one sensor 20, 21. These sensors 20, 21 can be configured to receive external information for adjusting an operation of the vibrotactile plush device 1. For example, the method can include providing a sensor 20, 21 configured to detect a relative orientation or position of the vibrotactile plush device 1 and can instruct the vibrotactile plush device 1 to shut off or cease outputting vibrations from the at least one transducer 7 if the vibrotactile contact surface 2 is no longer in contact with the body of the user. Such a sensor can act as a power-saving feature of the vibrotactile plush device 1 when the vibrotactile features of the vibrotactile plush device 1 are not actively being used.

In closing, regarding the exemplary embodiments of the present invention as shown and described herein, it will be appreciated that a vibrotactile cuddle device is disclosed. Because the principles of the invention may be practiced in a number of configurations beyond those shown and described, it is to be understood that the invention is not in any way limited by the exemplary embodiments, but is generally directed to a vibrotactile cuddle device and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art, or at least encompassing a range of plus or minus ten percent above and below the value of the stated characteristic, item, quantity, parameter, property, or term.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that any logic code, programs, modules, processes, and/or methods disclosed herein, along with the order in which the respective elements of any such method are performed, are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment. Additionally, the various illustrative logical blocks, modules, methods, and algorithm processes and sequences described in connection with the embodiments disclosed herein can be implemented as electronic hardware, computer software, or combinations of both. To clearly illustrate this interchangeability of hardware and software, various illustrative components, blocks, modules, and process actions have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of this document.

The phrase “non-transitory,” in addition to having its ordinary meaning, as used in this document means “enduring or long-lived.” The phrase “non-transitory computer readable medium,” in addition to having its ordinary meaning, includes any and all computer readable mediums, with the sole exception of a transitory, propagating signal. This includes, by way of example and not limitation, non-transitory computer-readable mediums such as register memory, processor cache and random-access memory (“RAM”).

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

All of the material in this patent document issue subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

What is claimed is:

1. A vibrotactile soothing device comprising:
a padding material defining a padding recess in a planar bottom surface of the padding material, the bottom surface of the padding material sized and configured for being placed in contact with a body of a user;
a sound barrier material positioned within the padding recess;
a planar, non-padded vibrotactile material spanning an opening of the padding recess such that an exterior surface of the planar, non-padded vibrotactile material lies in the same plane as the bottom surface of the padding material and a bottom edge of the sound barrier material, the planar, non-padded vibrotactile material cooperating with the bottom surface of the padding material and the bottom edge of the sound barrier material to form a continuous planar surface for being placed in contact with the body of the user;
a rigid plastic housing positioned within the padding recess so as to sandwich the sound barrier material between the housing and the padding material, a side wall of the housing in abutting contact with an opposing interior surface of the planar, non-padded vibrotactile material; and
at least one electroacoustic transducer positioned within the housing and coupled to an interior surface of the side wall of the housing, such that the side wall of the housing is sandwiched between the at least one electroacoustic transducer and the planar, non-padded vibrotactile material, the at least one electroacoustic transducer configured for selectively emitting sound vibrations through each of the side wall of the housing and the planar, non-padded vibrotactile material;
whereby, during use of the device, with the bottom surface of the padding material positioned so that the exterior surface of the planar, non-padded vibrotactile material is in contact with the body of a user, the planar, non-padded vibrotactile material and rigid plastic housing cooperate to maximize sound vibrations produced by the electroacoustic transducer, thereby soothing the user.

2. The vibrotactile soothing device of claim 1, further comprising a padding cover configured for substantially encapsulating the padding material and the planar, non-padded vibrotactile material.

3. The vibrotactile soothing device of claim 1, further comprising a controller in communication with the at least one electroacoustic transducer, the controller configured for transmitting an at least one output signal to be produced by the at least one electroacoustic transducer.

4. The vibrotactile soothing device of claim 3, further comprising at least one sensor for sensing external information and providing a signal to the controller in response to the external information, wherein the at least one output signal of the controller adjusts an operation of the soothing device in response to the external information.

5. The vibrotactile soothing device of claim 4, wherein the at least one sensor is configured for sensing at least one of an orientation of the vibrotactile soothing device, a position of the vibrotactile soothing device relative to the body of the user, a breathing pattern of the user, a breathing rate of the user, and a heartrate of the user.

6. The vibrotactile soothing device of claim 5, wherein the controller is configured for dynamically adjusting one or more of a sound, a vibration intensity or a vibration frequency produced by the at least one electroacoustic transducer to match a breathing pattern of the user as detected by the at least one external sensor, in response to the external information received by the at least one sensor.

7. The vibrotactile soothing device of claim 6, wherein:
the controller is configured for causing the at least one electroacoustic transducer to emit a first sound vibration in sync with the user inhaling; and
the controller is further configured for causing the at least one electroacoustic transducer to emit a second sound vibration in sync with the user exhaling, the second sound vibration being different than the first sound vibration;
whereby, the controller provides alternating inhalation and exhalation related sound vibrations at a frequency that matches the breathing rate of the user.

8. The vibrotactile soothing device of claim 6, wherein the controller is configured for dynamically adjusting the vibration frequency produced by the at least one electroacoustic transducer so as to be progressively slower, thereby causing the breathing rate of the user to progressively slow as well.

9. The vibrotactile soothing device of claim 4, wherein the at least one sensor is positioned within the padding material.

10. The vibrotactile soothing device of claim 4, wherein the at least one sensor is positioned on a separate wearable device configured for removable engagement with the user, the at least one sensor in wireless communication with the controller.

11. The vibrotactile soothing device of claim 10, wherein the wearable device is configured as one of a bracelet, an arm band, a smartwatch or a fitness tracker.

12. The vibrotactile soothing device of claim 3, wherein the controller is configured for transmitting an at least one further output signal to be produced by an at least one peripheral device.

13. The vibrotactile soothing device of claim 12, wherein the at least one peripheral device is a pair of headphones, an at least one earbud, a pair of bone conduction headphones, or an at least one speaker.

14. The vibrotactile soothing device of claim 12, wherein the at least one further output signal is an audio signal comprising one or more of music, sounds or speech.

15. The vibrotactile soothing device of claim 3, wherein the controller provides an at least one indicator light positioned and configured for assisting the user with operating the vibrotactile soothing device in low light environments.

16. The vibrotactile soothing device of claim 3, wherein the controller is configured for receiving a user input to customize at least one of a sound, a vibration pattern, a vibration intensity, or a vibration frequency generated by the at least one electroacoustic transducer.

17. The vibrotactile soothing device of claim 1, wherein the planar, non-padded vibrotactile material comprises at least one of a microcellular elastomer, a polyurethane, a rubber, or a microcellular polyurethane.

18. The vibrotactile soothing device of claim 1, wherein the planar, non-padded vibrotactile material has a thickness of approximately ⅛ of an inch.

19. A vibrotactile soothing device comprising:
a padding material defining a padding recess in a planar bottom surface of the padding material, the bottom surface of the padding material sized and configured for being placed in contact with a body of a user;
a sound barrier material positioned within the padding recess;
a planar, non-padded vibrotactile material spanning an opening of the padding recess such that an exterior surface of the planar, non-padded vibrotactile material lies in the same plane as the bottom surface of the padding material and a bottom edge of the sound barrier material, the planar, non-padded vibrotactile material cooperating with the bottom surface of the padding material and the bottom edge of the sound barrier material to form a continuous planar surface for being placed in contact with the body of the user;
a rigid plastic housing positioned within the padding recess so as to sandwich the sound barrier material between the housing and the padding material, a side wall of the housing in abutting contact with an opposing interior surface of the planar, non-padded vibrotactile material; and
at least one electroacoustic transducer positioned within the housing and coupled to an interior surface of the side wall of the housing, such that the side wall of the housing is sandwiched between the at least one electroacoustic transducer and the planar, non-padded vibrotactile material, the at least one electroacoustic transducer configured for selectively emitting sound vibrations through each of the side wall of the housing and the planar, non-padded vibrotactile material;
at least one sensor positioned and configured for sensing at least one of an orientation of the vibrotactile soothing device, a position of the vibrotactile soothing device relative to the body of the user, a breathing pattern of the user, a breathing rate of the user, and a heartrate of the user;
a controller in communication with each of the at least one electroacoustic transducer and sensor, the controller configured for transmitting an at least one output signal to be produced as sound vibrations by the at least one electroacoustic transducer; and
the controller further configured for dynamically adjusting one or more of a sound, a vibration intensity or a vibration frequency of the sound vibrations produced by the at least one electroacoustic transducer so as to match a breathing pattern of the user as detected by the at least one external sensor;
whereby, during use of the device, with the bottom surface of the padding material positioned so that the exterior surface of the planar, non-padded vibrotactile material is in contact with the body of a user, the planar, non-padded vibrotactile material and rigid plastic housing cooperate to maximize sound vibrations produced by the electroacoustic transducer, thereby soothing the user.

20. A vibrotactile soothing device comprising:
a padding material defining a padding recess in a planar bottom surface of the padding material, the bottom surface of the padding material sized and configured for being placed in contact with a body of a user;
a sound barrier material positioned within the padding recess;
a rigid, airtight housing positioned within the padding recess so as to sandwich the sound barrier material between the housing and the padding material, the housing providing a main compartment and a radiator tube in fluid communication with the main compartment;
a first end of the radiator tube connected to the main compartment, with an opposing second end of the radiator tube being open-ended and positioned proximal to but spaced apart from the bottom surface of the padding material and a bottom edge of the sound barrier material;
at least one electroacoustic transducer positioned within the housing and comprising an active speaker coupled to an interior surface of a side wall of the main compartment; and
a substantially planar passive radiator engaged with the second end of the radiator tube and comprising a weighted disc surrounded by a diaphragm constructed out of a non-padded vibrotactile material, the passive radiator spanning the open second end of the radiator tube such that an exterior surface of the diaphragm lies in the same plane as the bottom surface of the padding material and the bottom edge of the sound barrier material, the diaphragm cooperating with the bottom surface of the padding material and the bottom edge of the sound barrier material to form a continuous planar surface for being placed in contact with the body of the user;
whereby, during use of the device, with the bottom surface of the padding material positioned so that the exterior surface of the diaphragm is in contact with the body of a user, the active speaker of the at least one electroacoustic transducer selectively emits sound vibrations which displaces air within the housing and drives the passive radiator to transfer sound vibrations to the diaphragm, thereby maximizing sound vibrations and soothing the user.
